(19) United States
(12) Patent Application Publication
(10) Pub. No.: US 2021/0204817 A1
(43) Pub. Date: Jul. 8, 2021

(54) PHOTOACOUSTIC IMAGING DEVICE AND METHOD

(71) Applicant: OLYMPUS CORPORATION, Tokyo (JP)

(72) Inventors: Yoshiaki MURAYAMA, Tokyo (JP); Atsushi DOI, Tokyo (JP); Kentaro IMOTO, Tokyo (JP)

(73) Assignee: OLYMPUS CORPORATION, Tokyo (JP)

(21) Appl. No.: 17/207,019

(22) Filed: Mar. 19, 2021

Related U.S. Application Data
(63) Continuation of application No. PCT/JP2018/035472, filed on Sep. 25, 2018.

(51) Int. Cl. A61B 5/00

(52) U.S. Cl. CPC A61B 5/0095; A61B 5/0073

(57) ABSTRACT

A photoacoustic imaging device includes an excitation light irradiation unit that irradiates the same position of a specimen with two or more beams of pulsed excitation light separated by an irradiation time interval, a detector that detects an acoustic wave generated at the position irradiated with the excitation light by the excitation light irradiation unit, a generator that generates an image of the specimen based on the detected acoustic wave, and a controller that controls the irradiation time interval, the acoustic wave generated with a single beam of pulsed excitation light has multiple positive peaks that periodically occur, and the controller controls the irradiation time interval into a timing at which a second positive peak of the acoustic wave generated with the preceding excitation light is superimposed on a first positive peak of the acoustic wave generated with the subsequent excitation light.

### CROSS-REFERENCE TO RELATED APPLICATIONS

This is a continuation of International Application PCT/JP2018/035472 which is hereby incorporated by reference herein in its entirety.

### TECHNICAL FIELD

The present invention relates to a photoacoustic imaging device and method.

### BACKGROUND ART

A photoacoustic imaging device is known in which a specimen is irradiated with pulsed excitation light, and acoustic wave generated in the specimen is detected to thereby acquire an image of the specimen (see PTL 1, for example).

### CITATION LIST

### Patent Literature

{PTL 1}

Japanese Translation of PCT International Application, Publication No. 2011-519281

### SUMMARY OF INVENTION

According to an aspect of the present invention, provided is a photoacoustic imaging device including an excitation light irradiation unit that irradiates the same position of a specimen with two or more beams of pulsed excitation light separated by an irradiation time interval, a detector that detects an acoustic wave generated at the position irradiated with the excitation light by the excitation light irradiation unit, a generator that generates an image of the specimen based on the detected acoustic wave, and a controller that controls the irradiation time interval, wherein the acoustic wave generated with a single beam of pulsed excitation light has multiple positive peaks that periodically occur, and the controller controls the irradiation time interval into a timing at which a second positive peak of the acoustic wave generated with the preceding excitation light is superimposed on a first positive peak of the acoustic wave generated with the subsequent excitation light.

Additionally, according to another aspect of the present invention, provided is a photoacoustic imaging method including irradiating the same position of a specimen with two or more beams of pulsed excitation light separated by an irradiation time interval, detecting an acoustic wave generated at the position irradiated with the excitation light, generating an image of the specimen based on the detected acoustic wave, wherein the acoustic wave generated with a single beam of pulsed excitation light has multiple positive peaks that periodically occur, and adjusting the irradiation time interval into a timing at which a second positive peak of the acoustic wave generated with the preceding excitation light is superimposed on a first positive peak of the acoustic wave generated with the subsequent excitation light.

### BRIEF DESCRIPTION OF DRAWINGS

FIG. 1 is an entire configuration diagram showing a photoacoustic imaging device according to an embodiment of the present invention.

FIG. 2 is a diagram showing an example of change in intensity of an acoustic wave generated in a specimen over time, the specimen being irradiated with a single beam of pulsed laser light, by the photoacoustic imaging device of FIG. 1.

FIG. 3 is a diagram showing an example of change in intensity of an acoustic wave generated in a specimen over time, the specimen being irradiated with two beams of pulsed laser light, by the photoacoustic imaging device of FIG. 1.

FIG. 4 is a diagram showing an example of change in intensity of an acoustic wave over time that is detected when two acoustic waves of FIG. 3 are generated.

### DESCRIPTION OF EMBODIMENTS

Hereinafter, description will be made as to a photoacoustic imaging device 1 and a photoacoustic imaging method according to an embodiment of the present invention with reference to the drawings.

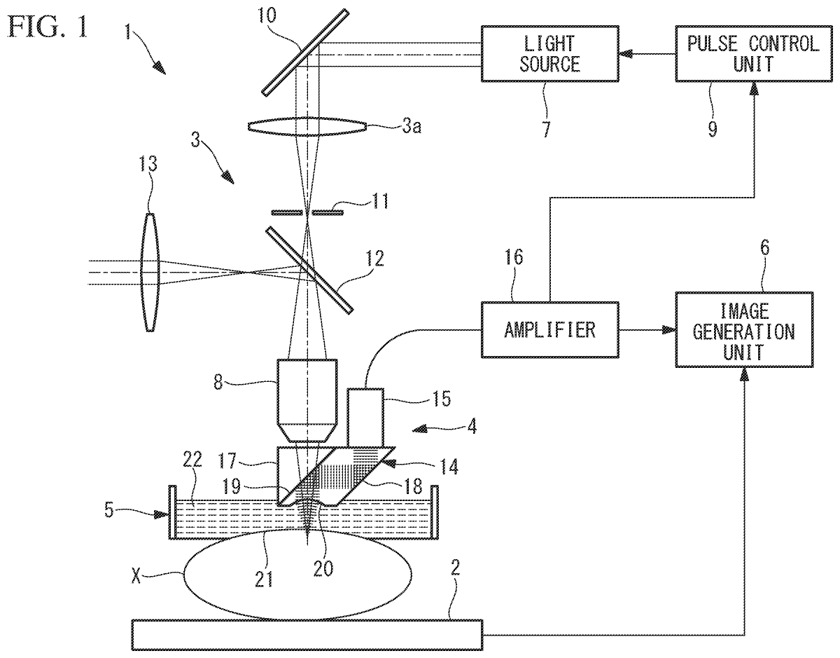

The photoacoustic imaging device 1 according to the present embodiment includes, as shown in FIG. 1, a stage 2 on which a specimen X is mounted, an excitation light irradiation unit 3 that irradiates the specimen X mounted on the stage 2 with excitation light, an acoustic wave detection unit 4 that detects an acoustic wave generated in the specimen X irradiated with the excitation light, a water tank 5 fixed to the stage 2, and an image generation unit 6 that generates an image based on the detected acoustic wave.

The stage 2 can move the mounted specimen X in a three-dimensional direction. That is, the stage 2 is moved upward and downward in a vertical direction relative to an objective lens 8 described later, so that a focal position of the objective lens 8 can be moved in a depth direction of the specimen X. Furthermore, the stage 2 is moved in a horizontal direction relative to the objective lens 8, so that a position to be irradiated with laser light can be adjusted in the horizontal direction.

The excitation light irradiation unit 3 includes a light source 7 that generates pulsed laser light, a pulse control unit 9 that controls the light source 7, and the objective lens 8 that condenses the laser light generated in the light source 7, on a region of interest of the specimen. Furthermore, the excitation light irradiation unit 3 irradiates the same position of the specimen X with two or more beams of pulsed laser light separated by a predetermined irradiation time interval.

In the drawing, reference sign 3a denotes a condenser lens, numeral 10 denotes a mirror, numeral 11 denotes a pinhole, numeral 12 denotes a beam splitter, and numeral 13 denotes an eyepiece lens.

The acoustic wave detection unit 4 includes a branch element 14 that branches the acoustic wave generated in the specimen X from an optical path of the laser light, and an acoustic wave transducer 15 disposed in contact with an upper surface of the branch element 14. The acoustic wave transducer 15 outputs intensity of the detected acoustic wave as a voltage signal. In the drawing, numeral 16 denotes an amplifier that amplifies the voltage signal outputted from the acoustic wave transducer 15.

The image generation unit 6 generates the image based on the voltage signal of the acoustic wave intensity amplified by the amplifier 16 and positional information of the stage 2.

The branch element 14 has a configuration of a triangular prism 17 combined with a parallelogram prism 18, and is disposed close to a tip of the objective lens 8.

An inclined surface of the triangular prism 17 and an inclined surface of the parallelogram prism 18 that are arranged adjacent to each other are separated by a liquid disposed between both the surfaces, that is, a nonvolatile liquid with a matched optical refractive index and a low acoustic impedance, such as a thin layer of low molecular weight silicone oil. This layer forms a branch surface 19.

An upper surface of the triangular prism 17 disposed facing and below the tip of objective lens 8 is disposed orthogonally to an optical axis of the objective lens 8.

Consequently, the laser light that exits from the objective lens 8, to enter the triangular prism 17 is transmitted by the branch surface 19 and is emitted from a lower surface of the parallelogram prism 18 to outside the branch element 14. In this case, the laser light is inhibited from being refracted in the upper surface of the triangular prism 17 and the branch surface 19, and the specimen X vertically below the objective lens is straightly irradiated with the laser light emitted from the objective lens 8.

In the present embodiment, the laser light exits from the lower surface of the parallelogram prism 18, and the acoustic wave enters the lower surface. In this lower surface, a recess (an acoustic lens) 20 that collects the entering acoustic wave is provided. The acoustic wave that enters the branch element 14 from the lower surface of the parallelogram prism 18 is collected in the recess 20 to enter the parallelogram prism 18, reflected by the branch surface 19 and a facing surface parallel to the branch surface 19, in the parallelogram prism 18, and then exits from an upper surface of the parallelogram prism 18 adjacent to the facing surface to an interior of the branch element 14. On this upper surface, the acoustic wave transducer 15 is disposed, so that the acoustic wave can be detected.

The water tank 5 is a container in which water surface is formed and water 22 can be stored, and the water tank has a bottom surface provided with a membrane 21 that is deformable in contact with the specimen X. The membrane 21 is made of a material that can transmit the laser light and acoustic wave, such as silicone resin. The lower surface of the parallelogram prism 18 is soaked in the water surface of the stored water 22 in a state where any bubbles are not formed. Consequently, the laser light can be prevented from being refracted in the lower surface, and the acoustic wave can enter the branch element 14 without any loss. Furthermore, the water tank 5 and the branch element 14 can be relatively moved in a state where the lower surface of the parallelogram prism 18 remains soaked in the water surface.

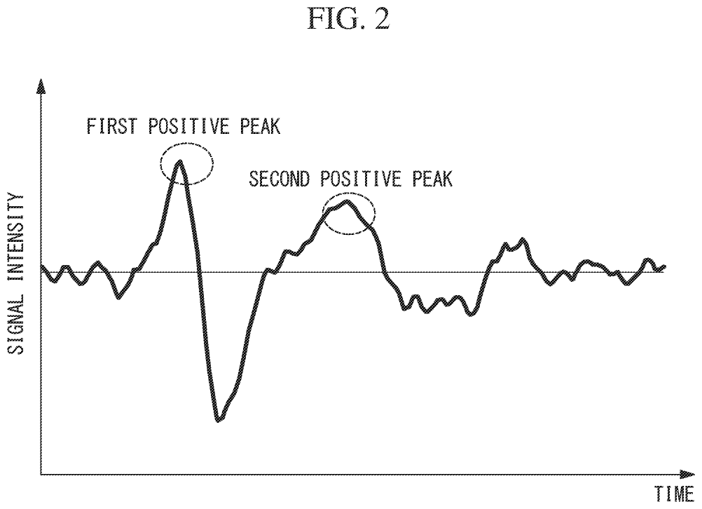

Here, upon irradiation of the specimen X with a single beam of pulsed laser light, the acoustic wave, generated at the position of the specimen irradiated with the laser light, generally has multiple positive peaks that periodically occur separated by a time interval as shown in FIG. 2. An occurrence period of the positive peaks varies with a type of specimen X, a wavelength of the laser light and the like, and hence, the period may be measured and stored in advance.

In the present embodiment, as the light source 7, used is a light source capable of emitting the pulsed laser light at a time interval that is ¼ or less of a time interval of the positive peak of the acoustic wave that is measured in advance as described above.

Then, the pulse control unit 9 controls an irradiation time interval of two beams of pulsed laser light with which the same position of the specimen X is irradiated at the irradiation time interval by the excitation light irradiation unit 3.

Specifically, the pulse control unit 9 is triggered by the voltage signal transmitted from the acoustic wave transducer 15 through the amplifier 16, to control the irradiation time interval into a timing at which, in the two beams of pulsed laser light separated by the time interval, a second positive peak, from the beginning, of the acoustic wave generated in the specimen X irradiated with preceding laser light is superimposed on a first positive peak of the acoustic wave generated in the specimen X irradiated with subsequent laser light.

Further specifically, the pulse control unit 9 thins out the pulsed laser light emitted from the light source 7, to thereby selectively emit, from the light source 7, two beams of pulsed laser light closest to the above timing. The light source 7 is capable of emitting the pulsed laser light at the time interval that is ¼ or less of the timing, and hence, any time interval between two beams of pulsed laser light is very likely to be close to the above timing. Here, the superimposition of two peaks of the acoustic wave includes partial overlap, in addition to complete match.

Next, description will be made as to a photoacoustic imaging method in which the photoacoustic imaging device 1 according to the present embodiment is used.

To perform imaging of the specimen X by use of the photoacoustic imaging device 1 according to the present embodiment, the specimen X, such as a mouse, is mounted on the stage 2, and as shown in FIG. 1, the water tank 5 is fixed to the stage 2 from above the specimen X, in a state where the membrane 21 of the bottom surface of the water tank 5 filled with the water 22 is in close contact with a surface of the specimen X.

Then, when the stage 2 is raised to a position where the focal position of the objective lens 8 is disposed at a desirable position in the specimen X, the pulsed laser light is generated from the light source 7. The light source 7 is controlled by the pulse control unit 9, to emit two beams of pulsed laser light separated by a predetermined irradiation time interval. The emitted laser light beams pass through the condenser lens 3a, the pinhole 11 and the beam splitter 12, to be condensed by the objective lens 8, and the laser light beams are transmitted by the branch element 14 and the water 22 in the water tank 5, to enter the same position of the specimen X.

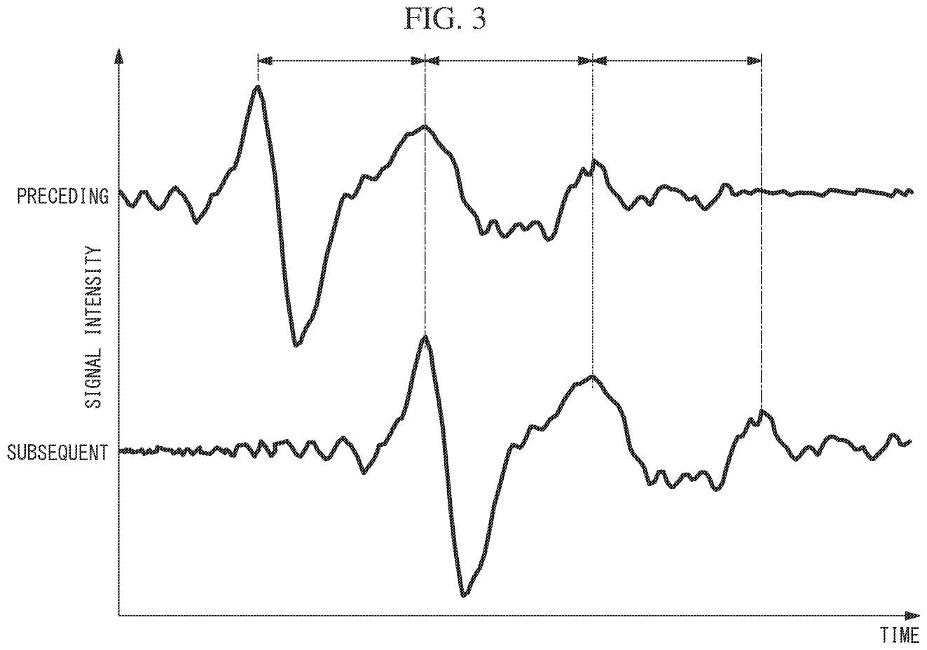

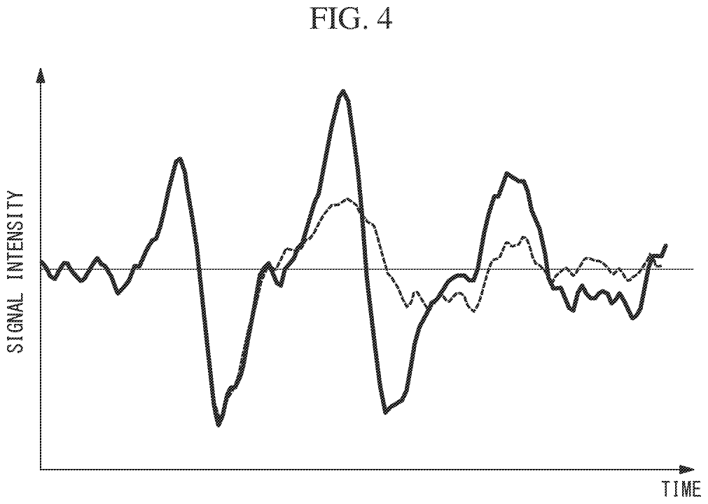

In this case, the acoustic wave generated in the specimen X irradiated with the preceding pulsed laser light and the acoustic wave generated in the specimen X irradiated with the subsequent pulsed laser light have equal waveforms as shown in FIG. 3. The generated acoustic wave shifts from the other generated acoustic wave by the irradiation time interval between two beams of pulsed laser light. As a result, as shown in FIG. 4, the second positive peak of the preceding pulsed laser light is superimposed on the first positive peak of the subsequent pulsed laser light, to increase a peak value.

The generated acoustic wave propagates from an interior of the specimen X to the water 22 in the water tank 5, to enter the branch element 14, and is reflected by the branch surface 19 and a facing surface in the parallelogram prism 18 of the branch element 14, to be detected by the acoustic wave transducer 15. Any air layer is not present in a path from a position where the acoustic wave is generated to the acoustic wave transducer 15, and hence, the acoustic wave propagates without being attenuated, and can be efficiently detected. Then, the intensity of the detected acoustic wave is associated with the position irradiated with the laser light, by the image generation unit 6, and the image of the specimen X is accordingly generated.

In this case, according to the photoacoustic imaging device 1 and the photoacoustic imaging method of the present embodiment, the peak value of the generated acoustic wave is increased without increasing the intensity of the laser light with which the specimen X is to be irradiated. Consequently, there is an advantage that an image with a satisfactory S/N ratio can be obtained, without increasing damages on the specimen X.

That is, if intensity of laser light with which a living body is to be irradiated is increased, energy of the laser light is spatially and temporally concentrated on the specimen X, to raise a temperature of the specimen, which causes damages to the specimen X. On the other hand, according to the photoacoustic imaging device 1 and method of the present embodiment, energy is released from the specimen X between the positive peaks of the acoustic wave, and hence, the acoustic wave with large intensity can be detected while suppressing temperature rise due to the concentration of the energy.

Note that in the present embodiment, the light source 7 capable of emitting the pulsed laser light in a constant period is used, and the laser light to be emitted from the light source 7 is thinned out. Consequently, two beams of pulsed laser light are emitted at timings that approximate to an occurrence period of the positive peaks of the acoustic wave. Alternatively, an emission period may be adjusted by using the light source 7 capable of emitting the pulsed laser light in an arbitrary period, and accordingly, two beams of pulsed laser light may be emitted at the timings that match the occurrence period of the positive peaks of the acoustic wave.

In this case, the occurrence period of the positive peaks may be calculated based on the waveforms of the detected acoustic waves, and the light source 7 may be controlled based on the calculated occurrence period.

Furthermore, in the present embodiment, the stage 2 on which the specimen X is mounted is moved in a three-dimensional direction, to move the specimen X and the water tank 5 relative to the acoustic wave detection unit 4. Alternatively, the stage 2 may be fixed, and the acoustic wave detection unit 4 may be moved in the three-dimensional direction.

The water 22 is illustrated as an acoustic wave propagation medium, but any other acoustic wave propagation medium may be adopted.

The above-described embodiment also leads to the following aspects.

According to an aspect of the present invention, provided is a photoacoustic imaging device including an excitation light irradiation unit that irradiates the same position of a specimen with two or more beams of pulsed excitation light separated by an irradiation time interval, an acoustic wave detection unit that detects an acoustic wave generated at the position irradiated with the excitation light by the excitation light irradiation unit, an image generation unit that generates an image of the specimen based on the detected acoustic wave, and a pulse control unit that controls the irradiation time interval of the excitation light with which the specimen is to be irradiated by the excitation light irradiation unit, wherein the acoustic wave generated with a single beam of pulsed excitation light has multiple positive peaks that periodically occur, and the pulse control unit controls the irradiation time interval into a timing at which a second positive peak of the acoustic wave generated with the preceding excitation light is superimposed on a first positive peak of the acoustic wave generated with the subsequent excitation light.

According to the present aspect, if the specimen is irradiated with the pulsed excitation light, the acoustic wave is generated at the irradiated position of the specimen, and is detected by the acoustic wave detection unit. In the image generation unit, a size of the acoustic wave at each position of the specimen is measured to be arranged, so that an acoustic wave image of the specimen can be generated.

In this case, the acoustic wave generated in response to the irradiation with the pulsed excitation light has a waveform in which the multiple positive peaks are arranged in a period depending on the specimen and the acoustic wave detection unit. The pulse control unit controls the irradiation time interval of two or more beams of pulsed excitation light into the timing at which the second positive peak of the acoustic wave generated with the preceding excitation light is superimposed on the first positive peak of the acoustic wave generated with the subsequent excitation light. Consequently, a peak value of the acoustic wave at time of the second positive peak to the preceding excitation light can be a larger value to which two peak values of the acoustic wave are added. Therefore, intensity of entering excitation light does not have to be increased, and an image with a satisfactory S/N ratio can be acquired without increasing damages on the specimen.

In the above aspect, the excitation light irradiation unit may include a light source capable of emitting the excitation light at an emission time interval that is ¼ or less of the irradiation time interval, and the pulse control unit may control the light source, to thin out the excitation light.

According to this configuration, even when the period of the positive peak of the acoustic wave generated in the specimen is not in a relation of being an integer multiple of the emission time interval of the pulsed excitation light from the light source, the second positive peak of the acoustic wave generated with the preceding excitation light can be superimposed on the first positive peak of the acoustic wave generated with the subsequent excitation light to a certain degree, only by thinning out the excitation light from the light source by the pulse control unit. Thus, intensity of the detected acoustic wave can be increased.

Furthermore, in the above aspect, the excitation light irradiation unit may include a light source in which an emission time interval is adjustable, and the pulse control unit may control the emission time interval into a timing that matches a period of the acoustic wave.

According to this configuration, the second positive peak of the acoustic wave generated with the preceding excitation light can be accurately matched with the first positive peak of the acoustic wave generated with the subsequent excitation light, and the intensity of the detected acoustic wave can be effectively increased.

Additionally, according to another aspect of the present invention, provided is a photoacoustic imaging method including irradiating the same position of a specimen with two or more beams of pulsed excitation light separated by an irradiation time interval, detecting an acoustic wave generated at the position irradiated with the excitation light, generating an image of the specimen based on the detected acoustic wave, wherein the acoustic wave generated with a single beam of pulsed excitation light has multiple positive peaks that periodically occur, and adjusting the irradiation time interval into a timing at which a second positive peak of the acoustic wave generated with the preceding excitation light is superimposed on a first positive peak of the acoustic wave generated with the subsequent excitation light.

### REFERENCE SIGNS LIST

1 photoacoustic imaging device
3 excitation light irradiation unit
4 acoustic wave detection unit
6 image generation unit
7 light source
9 pulse control unit
X specimen

1. A photoacoustic imaging device comprising:
an excitation light irradiation unit that irradiates the same position of a specimen with two or more beams of pulsed excitation light separated by an irradiation time interval,
a detector that detects an acoustic wave generated at the position irradiated with the excitation light by the excitation light irradiation unit,
a generator that generates an image of the specimen based on the detected acoustic wave, and
a controller that controls the irradiation time interval,
wherein the acoustic wave generated with a single beam of pulsed excitation light has multiple positive peaks that periodically occur, and
the controller controls the irradiation time interval into a timing at which a second positive peak of the acoustic wave generated with the preceding excitation light is superimposed on a first positive peak of the acoustic wave generated with the subsequent excitation light.

2. The photoacoustic imaging device according to claim 1, wherein the excitation light irradiation unit comprises a light source configured to emit the excitation light at an emission time interval that is ¼ or less of the irradiation time interval, and
the controller controls the light source, to thin out the excitation light.

3. The photoacoustic imaging device according to claim 1, wherein the excitation light irradiation unit comprises a light source in which an emission time interval is adjustable, and
the controller controls the emission time interval into a timing that matches a period of the acoustic wave.

4. A photoacoustic imaging method comprising:
irradiating the same position of a specimen with two or more beams of pulsed excitation light separated by an irradiation time interval,
detecting an acoustic wave generated at the position irradiated with the excitation light,
generating an image of the specimen based on the detected acoustic wave, wherein the acoustic wave generated with a single beam of pulsed excitation light has multiple positive peaks that periodically occur, and
adjusting the irradiation time interval into a timing at which a second positive peak of the acoustic wave generated with the preceding excitation light is superimposed on a first positive peak of the acoustic wave generated with the subsequent excitation light.
